(19) United States
(12) Patent Application Publication
(10) Pub. No.: US 2023/0000341 A1
(43) Pub. Date: Jan. 5, 2023

(54) OCT-BASED, SPATIALLY RESOLVED TRANSMISSION MEASUREMENT OF THE EYE

(71) Applicant: Haag-Streit AG, Köniz (CH)

(72) Inventor: Lucio ROBLEDO, Bern (CH)

(73) Assignee: Haag-Streit AG, Köniz (CH)

(21) Appl. No.: 17/781,588

(22) PCT Filed: Jan. 17, 2020
(86) PCT No.: PCT/EP2020/051189

§ 371 Date: Jun. 1, 2022

(51) Int. Cl. A61B 3/10; G01B 9/02091

(52) U.S. Cl. CPC A61B 3/102; G01B 9/02091

(57) ABSTRACT

A method for measuring at least one parameter indicative of the optical transmission quality of the eye, such as information on absorptive or scattering structures that affect the propagation of light between the cornea and the retina and/or information on the imaging quality, e.g., the point-spread-function of the eye. The method includes recording a plurality of optical coherence tomography A-scans for different cornea locations xi, yi of the eye by an optical coherence tomography device and a scanner. For each A-scan, a reflection value at the retina of the eye is determined. The reflection values can then be combined, e.g., for displaying an image of the eye's transmission quality as a function of xi, yi or, by Fourier analysis, for determining the point spread function of the eye.

### TECHNICAL FIELD

The invention relates to a method for measuring at least one parameter indicative of an optical transmission quality of the eye, such as information on absorptive or scattering structures that affect the propagation of light between the cornea and the retina and/or information on the imaging quality, e.g. the point-spread-function (PSF) of the eye.

### BACKGROUND ART

EP 2710950 describes a method for measuring intraocular scattering and, in particular, the point-spread-function (PSF) of the eye by shining an annular or circular light beam into the eye and by measuring its reflection from the retina.

In such a method, complex measures are required for eliminating reflection effects from the anterior part of the eye and for deriving the PSF of the eye.

### DISCLOSURE OF THE INVENTION

The problem to be solved by the present invention is to provide a method of this type that is able to reliably measure at least one parameter indicative of the optical transmission quality of the eye.

This problem is solved by the method and device of the independent claims.

Accordingly, the method comprises at least the following steps:
Recording a plurality of OCT A-scans for different lateral cornea locations xi, yi of the eye: In other words, a plurality of optical coherence tomography measurements are carried out by means of light beams sent through distinct cornea locations.
For each of said A-scans, identifying a reflection value ri at the retina of the eye: This reflection value is indicative of the amount of light that is reflected from the retina and returned into the OCT measurement system.
Determining the parameter(s) using said reflection values ri and said locations xi, yi: In other words, reflection values ri and their coordinates xi, yi are processed for determining the parameter.

Hence, optical coherence tomography data can be recorded for each A-scan. This allows to easily distinguish reflection values originating from the anterior part of the eye and from the retina, i.e. to isolate the reflection values ri from the retina. The reflection value ri depends on the eye's transmission properties along the respective A-scan at location xi, yi, which allows to obtain a spatially resolved indication how well the eye transmits light along the probe beam of A-scan i.

This information can be used for determining a large number of different parameters. Some examples include:
The parameter can be descriptive of at least one aspect of the point-spread-function PSF of the eye. For example, the method can comprise the step of determining, using the reflection values ri, a one- or two-dimensional representation of the point-spread-function, and/or it may deliver a characteristic of the same, such as its half-width along one or more directions.
The parameter can be descriptive of absorbing and/or scattering structures in the anterior segment of the eye. For example, the method can comprise the step of determining, using the reflection values ri, the location and/or spatial extent, in particular the location along xi and/or yi, of absorbing and/or scattering structures in the anterior segment of the eye, e.g. by representing the reflection values ri as an image in xi-yi-space.

Advantageously, the plurality of A-scans includes a first plurality of A-scans, advantageously at least 10 A-scans, in particular at least 100 A-scans, having parallel directions of incidence. In other words, the A-scans differ by their locations xi, yi but not by the direction of the light beams outside the eye as it impinges on the cornea. This allows to record the eye's transmission properties for light coming from a given direction. Further, for an eye accommodated to infinity, all such A-scans will substantially be incident on a common location of the retina, thereby providing better robustness of the measurement against spatially varying retina reflection.

In particular, the “parallel direction of incidence” may be parallel to the eye's visual axis, which allows to record the transmission properties along the patient's natural viewing direction.

In this context, “parallel” advantageously is understood to encompass a parallelism within 5°, in particular within 1°.

Advantageously, the A-scans include a plurality of A-scans, advantageously at least 10 A-scans, in particular at least 100 A-scans, that do not overlap at a cornea of the eye. In other words, these A-scans enter the eye at different locations xi, yi, allowing to record information with good spatial resolution.

In this context, advantageously, two A-scans do not overlap if their center distances on the cornea are larger than their half-width-diameter. The “half-width-diameter” is the diameter, in the x-y-plane perpendicular to the direction of the A-scan outside the eye, over which the intensity of the light used for the A-scan drops by 50%.

In another important embodiment, the probe beams of at least part of the A-scans are focused onto an anterior part of the eye, i.e. the probe beams have their minimum diameter in this anterior part. This allows to spatially resolve scattering or structures in that part of the eye.

In this context, an A-scan is advantageously considered to be focused at an anterior part of the eye if the minimum diameter of its probe beam is located somewhere within 1 mm before the cornea and 5 mm behind the eye's lens.

Alternatively or in addition thereto, the focus may, for at least part of the probe beams, also be located between the posterior surface of the eye's lens and the retina. This may be useful to e.g. detect vitreous floaters.

In one embodiment, the invention comprises the step of displaying the reflection values ri as a function of the locations xi, yi. Hence, an image is displayed representing the reflection value ri as a function of xi and yi. For example, the image can comprise pixels, where the pixel coordinates are mapped to the coordinates xi, yi and the pixel color and/or brightness is a function of the reflection value ri. Such an image allows to locate the regions where light rays are poorly transmitted through the eye, e.g. due to scattering and/or absorption.

This e.g. allows to locate absorbing structures in the anterior section of the eye. Again, the anterior section of the eye may e.g. be a section between the cornea and a location 5 mm behind the eye's lens.

To achieve good lateral resolution for absorbing or scattering structures in the vitreous, it is advantageous to place the focus between the posterior lens surface and the retina.

In another embodiment, the invention comprises a Fourier analysis of the dataset ri(xi, yi). This Fourier analysis comprises at least the following steps:
Performing a Fourier transform on a dataset based on the reflection values ri: This dataset can e.g. be the dataset ri(xi, yi), with the Fourier transform carried at along at least one dimension of the xi-yi-space. It may e.g. also be ri(θxi, θyi), with θxi and θyi being the horizontal and vertical angles of the direction of propagation at the posterior side of the lens, in respect to the eye's optical or visual axis, of the probe beam entering the eye at xi, yi.
Deriving said at least one parameter from the result of the Fourier transform: For example, the result can be the Fourier components, which describe the point-spread-function of the eye along at least one direction, or it can be a parameter derived from the Fourier components, such as a width (e.g. the half-width) or a contrast (e.g. ratio of peak amplitude and noise floor) of the PSF in at least one direction.

Advantageously, a two-dimensional Fourier transform is used, which allows to assess the PSF (or a parameter thereof) in two dimensions.

Alternatively or in addition to using the Fourier transform, ray tracing may be used for calculating the PSF, which allows to take into account the eye's refractive structures and in particular their aberrations, as they can e.g. be determined by means of the OCT measurements.

The method may further comprise at least one of the following steps:
Determining the axial length of the eye between the pupil and the retina from the A-scans by means of optical coherence tomography: This data can be easily derived from the A-scans.
Determining the diameter of the pupil: This data can also be easily derived from the A-scans or by means of a calibrated microscope.

Further the data from the A-scans may be used to extract the topology of at least one structure of the eye. This structure may e.g. be at least one of the following:
the cornea,
the iris,
the anterior surface of the lens, and/or
the posterior surface of the lens.

In that case, the method can further include the step of determining the at least one parameter using the reflection values ri and the topology of the structure, e.g. using ray tracing calculus.

The invention also relates to an ophthalmologic device comprising
An optical coherence tomography interferometer: This OCT interferometer is used to record the A-scans.
A control unit structured and adapted to carry out the method described herein: This control unit is provided with suitable software and hardware for carrying out the steps of the invention. It may also comprise a display, storage, and/or data interface for displaying, storing, and/or transferring the data determined by the present techniques.

### BRIEF DESCRIPTION OF THE DRAWINGS

The invention will be better understood and objects other than those set forth above will become apparent when consideration is given to the following detailed description thereof. Such description makes reference to the annexed drawings, wherein:

FIG. 1 shows the schematic setup of an embodiment of an ophthalmologic device,

FIG. 2 shows an embodiment of a scan pattern,

FIG. 3 shows the reflection values obtained in an A-scan,

FIG. 4 shows a sectional view of the eye with the incoming light traces of two A-scans,

FIG. 5 shows the reflection values ri of the cornea as a function of xi, yi for four different eyes A, B, C, and D,

FIG. 6 shows the PSF (point-spread-function) as obtained from the reflection values ri of FIG. 5 for the eyes A, B, C, and D, and

FIG. 7 shows intensity values of the PSF of FIG. 6 along horizontal (PSF H. u) and vertical (PSF V, v) directions for the eyes A, B, C, and D.

(Note: All grayscale images in the figures have been half-toned for better reproducibility. Half-toning is typically not used when representing the images on an electronic display.)

### MODES FOR CARRYING OUT THE INVENTION

Device Overview

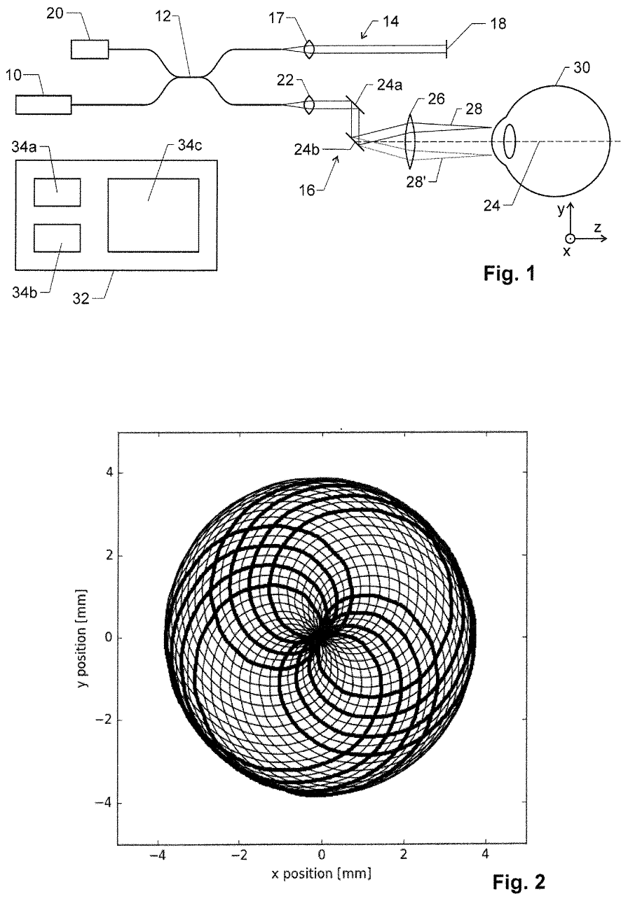

The ophthalmologic device of FIG. 1 is e.g. an ophthalmologic microscope with OCT capability.

It comprises an optical coherence tomography interferometer 10-26.

The interferometer has a light source 10, which, in the present embodiment, is a swept-source light source, i.e. it generates narrowband light that can be adjusted in wavelength.

The light from light source 10 passes a beam splitter 12, in particular a fiber beam splitter, and is sent into two interferometer arms 14, 16.

The first arm is the reference arm 14, which comprises a collimating lens 17 and mirror 18 at one end. Light impinging on mirror 18 is sent back into beam splitter 12 and from there, at least in part, to a light detector 20.

The second arm is the sample arm 16. It comprises collimation optics 22 for collimating the probe light coming from beam splitter 12. The light is then fed through two scanner mirrors 24a, 24b and an objective lens 26 for generating a probe beam 28. Depending on the position of the scanner mirrors 24a, 24b, probe beam 28 can be laterally offset in an x-y-plane perpendicular to the optical axis z of the device.

In the present embodiment, an interferometer generating telecentric probe beams 28 is used, i.e. the probe beams 28 for various x- and y-coordinates (such as beam 28 and beam 28′ in FIG. 1) are parallel to each other. This can be realized by placing the pivot point of the scanning system approximately at the back focal plane of lens 26. A telecentric scan geometry simplifies the analysis in the context of the techniques described below.

In the shown embodiment, the probe beams are shown to be focused on the anterior surface of the cornea, but they may also be focused on any other part of the eye 30 that is of particular interest. For the reasons mentioned above, the probe beams are advantageously focused on the anterior segment of the eye.

The focusing optics, e.g. the position and/or the power of lenses 22 and/or 26, may be adjustable to vary the position of the focus along the z-direction.

Probe beam 28 enters eye 30, where it is reflected or scattered by the structures of the eye. Light cast back from such structures is returned to beam splitter 12, where it can interfere with the light from reference arm 14, and from there, at least in part, to light detector 20.

The device of FIG. 1 is operated by recording a plurality of A-scans. For each such A-scan i, probe beam 28 is brought to a desired xi- and yi-position by means of the scanner mirrors 24a, 24b. Then, the central wavelength of light source 10 is tuned over a given wavelength range, which wavelength range is typically much broader than the spectral width of the light from light source 10. The light at light detector 20 is measured as a function of the central wavelength.

Spectral analysis, in particular a Fourier transform, of the signal from detector 20 can then be used for generating the reflection values of eye 30 along axis z for the given A-scan. Reflection values are meant to relate to reflected and scattered light as described above. As customary in OCT imaging, reflection values might be represented by values proportional to the reflected intensity or by values proportional to a logarithm of the reflected intensity or e.g. by other range-compressed values. In more general terms, a “reflection value” is indicative of the amount of light returned from a certain position along an A-scan. Advantageously, it may be linear to the amount of light or a logarithm thereof or any other function thereof.

This type of OCT measurement is known to the skilled person, and it is e.g. described in EP 3572765 and the references cited therein.

The device further comprises a control unit 32, which may e.g. be provided with a microprocessor 34a and with a memory 34b as well as with a display 34c. Memory 34b may hold the data as well as the program instructions required for carrying out the steps of the present method. Display 34c may e.g. be used for showing the data determined thereby and in particular for displaying plots or images as described below.

Advantageously, the measuring range (for a single A-scan) of the OCT interferometer 10-26 extends at least from the cornea to the retina of a typical eye. In other words, with a single A-scan (i.e. for an SS-OCT with a single sweep of the light source), depth-resolved information over at least 40 mm (in air) can be obtained. This allows to apply the techniques described in the following to be used over the whole axial eye length without the need to e.g. apply stitching for combining different measurements.

FIG. 2 shows an example for a scan pattern used in the measurement, i.e. it shows the locations of the probe beam 28 in the x-y-plane during the various A-scans. This type of pattern is described in EP 3021071. Other scan patterns can be used as well, such as the scan patterns e.g. described in EP 3217144 or U.S. Pat. No. 8,705,048.

A-Scan Analysis

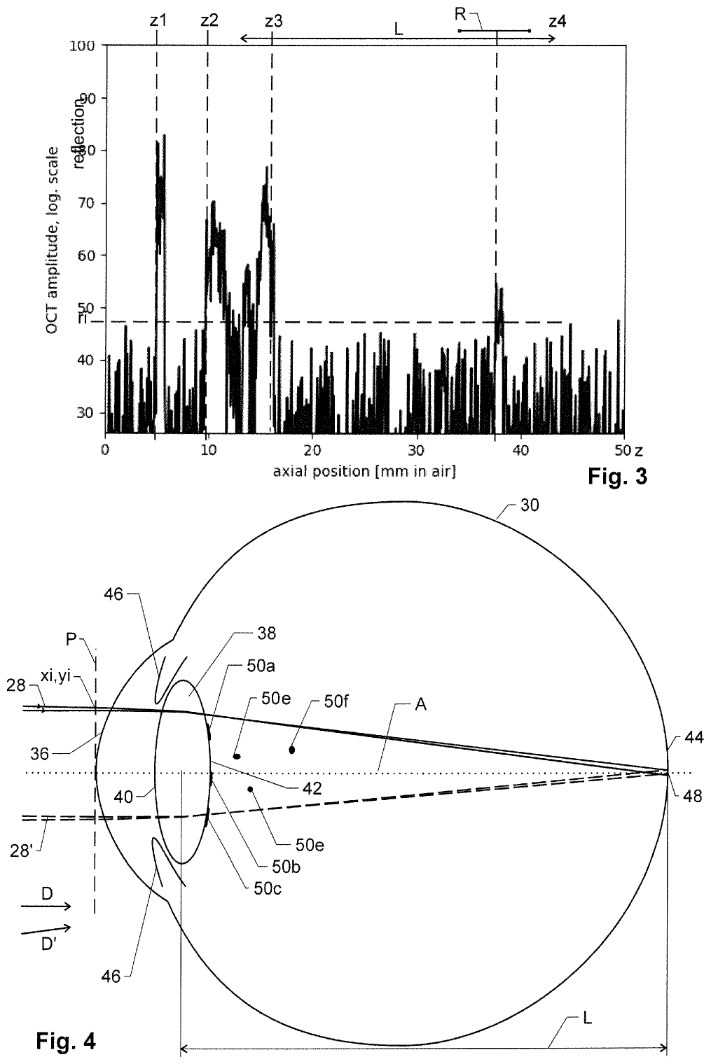

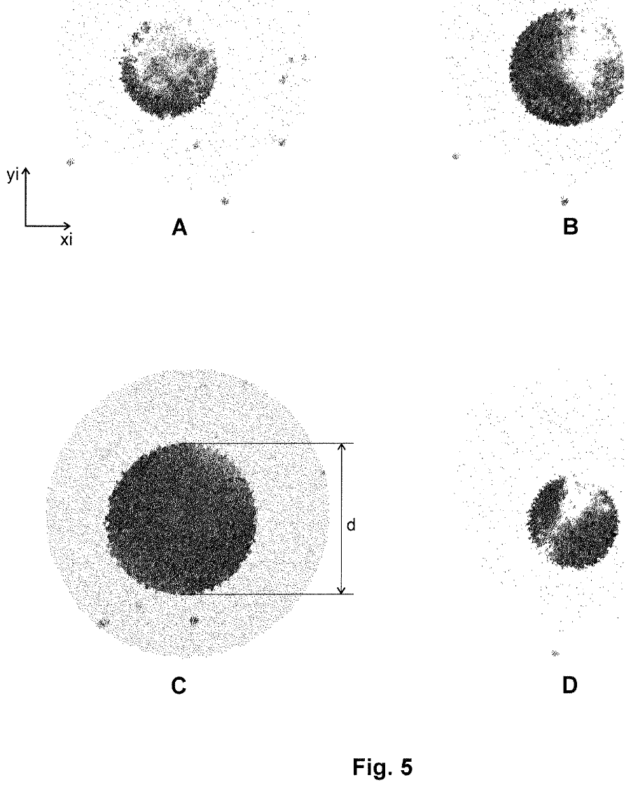

FIG. 3 shows the reflection values as obtained by means of OCT analysis for a single A-scan 28 (cf. FIG. 4) positioned at x=xi, y=yi in a plane P at the location of the apex of the cornea 36.

As known to the skilled person, the various structures of the eye generate different peaks in the reflection values corresponding to different depths z1, z2, z3 . . . A first major peak at a depth z1 may e.g. correspond to the (anterior surface of) the cornea 36, a second peak at z2 to the anterior surface 40 of the lens 38, a next peak at z3 to the posterior surface 42 of the lens 38, and a last peak at z4 to the retina 44.

The A-scans recorded in this manner can optionally be corrected for eye motion, e.g. by using at least the following steps:
1. Identifying reflections of at least one given eye-structure (such as the anterior corneal surface) in the A-scans.
2. Fitting a model descriptive of the shape of the structure and of the motion of the structure to the locations of the identified reflections. This model can e.g. have geometric parameters (such as the curvature) of the structure as well as motion parameters (such as the three-dimensional location and velocity in x-, y-, and z-coordinates).

The parameters obtained in the fitting step 2 can then be used for translating the OCT measurements, and in particular the incident coordinates xi, yi as well as the z-coordinates obtained from the A-scan, into a coordinate system that is fixed with the frame of the eye.

Suitable motion correction techniques are e.g. described in WO 2013/107649 or U.S. Pat. No. 7,452,077.

These steps allow to determine the location of various structures in the eye, such as cornea 36, the anterior and/or posterior surfaces 40, 42 of lens 38, and/or the anterior surface of the iris 46 and to identify their reflection values.

Transmission Analysis

As mentioned above, a reflection value of particular interest is the reflection value ri corresponding to the reflection of the probe beam of A-scan i at the retina 44.

This reflection value ri can e.g. be obtained by one of the following methods:
Determining a maximum of the reflection values in a region R around the expected z-location of the retina;
Integrating the reflection values over a given region R around the expected or determined z-location z4 of the retina. (The z-location of the retina may e.g. be determined from the z-location of the maximum reflection value in an expected z-location range R of the retina.
Fitting a model of a typical retina reflection to the reflection values at the expected z-location range R of the retina.

A more robust reflection value r′i can be obtained by combining values ri1, ri2, . . . , rin, of n A-scans i at points xi1/yi1, xi2/yi2, . . . xin/yin, having mutual distances smaller than a threshold d, e.g. with d<1 mm, <0.5 mm or <0.25 mm, by means of e.g. calculating an average, median, or weighted average of ri, ri2, . . . , rin.

The reflection value ri obtained in this manner is not only a function of the reflectivity of the retina but also a function of the transmission of the eye along the path of probe beam 28.

Hence, if the eye comprises scattering and/or absorbing structures along the path of probe beam 28, the reflection value ri decreases.

In a typical measurement, a plurality of A-scans i with i=1 . . . N (with N being at least 10, in particular at least 100, advantageously at least 1000) is performed. FIG. 4 shows the probe beams 28 and 28′ for two such A-scans.

Advantageously, the directions of incidence D of the probe beams outside the eye are parallel to each other and, advantageously, parallel to the eye's visual axis A.

For parallel probe beams 28, 28′ and an eye accommodated to infinity, the probe beams will all hit the retina 44 at a common location 48 (corresponding to the fovea if the direction of incidence of the A-scans outside the eye correspond to the eye's visual axis A).

Hence, the difference between reflection values ri for the retina for these two A-scans will primarily be due to the eye's different transmission for the two probe beams 28, 28′.

In other words, the reflection values ri of the retina describe how the transmission of the eye varies as a function of A-scan location xi, yi.

If, for example, there are local, scattering or absorbing structures 50a-50f in the anterior section of the eye, they can be detected and spatially resolved (at least in the directions x and y if not necessarily along z) by reviewing the reflection values ri as a function of scan location xi, yi.

For example, these structures may include scattering or absorbing structures 50a-50c at the posterior surface of the lens or scattering and/or absorbing structures 50d-50f in the anterior half of the eye behind the lens.

This is illustrated in FIG. 5, which shows the reflection values ri for different eyes as a function of the coordinates xi, yi, with black or dark regions of the figure denoting high reflection values ri and white or bright regions of the figure denoting low reflection values ri from the retina.

In each image, the pupil can be recognized easily. Positions where the A-scan hits the iris have low reflection values ri from the retina and are, therefore, white.

Eye C of FIG. 5 shows a consistently high reflection value ri from the retina within the pupil, indicating an eye with consistently good transmission.

Eyes A, B, and D show eyes where the transmission is impaired for some locations xi, yi, which is indicative of defects in the eye's anterior region.

It must be noted that the present techniques allows to detect not only scattering but also absorbing structures. The latter are notoriously hard to detect by other methods.

PSF Analysis

An analysis of the reflection values ri as a function of xi, yi allows to obtain an estimate of the eye's PSF.

The relevant techniques are e.g. described in Goodman J W, “Introduction to Fourier optics”, 2^{nd} edition (1996).

In particular, and assuming that the eye's lens and cornea provide perfect imaging only impaired by defects 50a-50f in the eye's anterior section, the PSF can be calculated by the Fourier transform FT of the modulation transfer function MTF of the anterior eye, i.e.

PSF=FT(MTF) (1)

The modulation transfer function can be estimated from the reflection values ri(xi, yi) as obtained by the measurements described in the section “PSF analysis” above. Advantageously the MTF is interpolated to a regular 2D grid since this allows to use the efficient FFT algorithm to perform the FT.

In particular, and in good approximation

PSF(u,v)=FT(ri(θxi, θyi)) (2)

with θxi, θyi being the angles of propagation on the posterior side of the lens, of probe beam for A-scan i and u, v being retina coordinates. The angles θxi, θyi are measured in to the axis A of the eye.

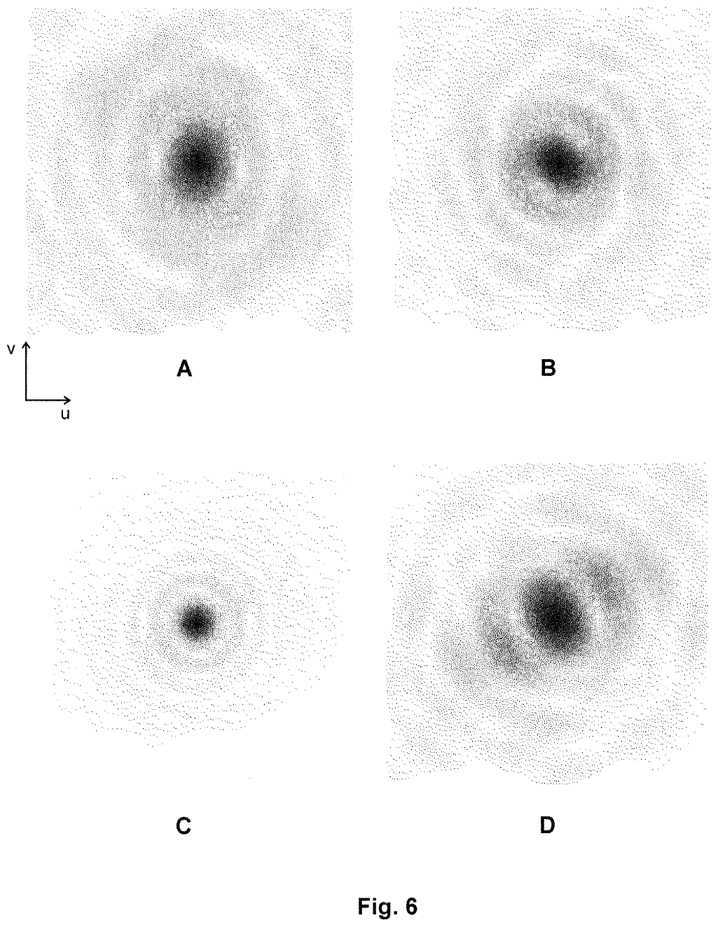

FIG. 6 shows examples of PSF(u, v) as calculated for from the reflection values ri(xi, yi) of the eyes of FIG. 5. As can be seen, eye C with its wide pupil and good, homogeneous transmission provides the best PSF, i.e. the PSF with smallest scatter, while the eyes A, B, and D have poorer imaging properties.

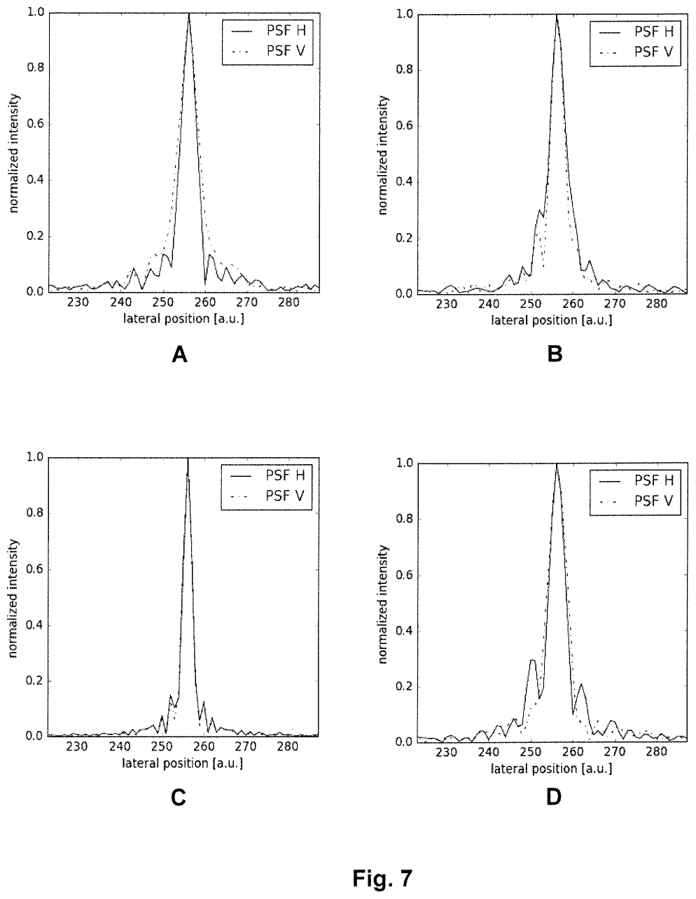

FIG. 7 shows the profiles of PSF(u) and PSF(v) in horizontal and vertical direction, again for the eyes A-D of FIG. 5.

For a quantitative analysis, the values θxi, θyi can be calculated from xi, yi using the axial length L of the eye. In this context, this axial length L may be defined as the distance, along axis A, between the center of lens 38 and retina 44. Alternatively, it may e.g. be defined as the distance, along axis A, between any other part of lens 38 and retina 44 or the distance between the apex of cornea 36 and retina 44.

In particular, the values θxi, θyi can be calculated using ray tracing techniques.

This axial length L of the eye can easily be determined from the OCT A-scans by determining the positions of the respective peaks in the A-scan spectra. In the example of FIG. 3, L is e.g. calculated from z4−(z2+z3)/2.

Hence, the present method advantageously comprises the step of using the axial length L in order to estimate a parameter descriptive of the absolute size of the PSF, such as a half-width of the PSF in horizontal and/or vertical direction.

In addition, for a quantitative analysis, the absolute values of xi, yi need to be known, e.g. from one or more of the following sources:
The scanning optics 24a, 24b may be calibrated to yield known displacements in respect to the axis of the system. In this case, absolute values of xi, yi can be derived from the settings of the scanning optics 24a, 24b for a given A-scan i.
In the OCT measurements, reflections from the iris can be identified, which allows to measure the diameter d of the iris (see e.g. FIG. 5, eye C) in the coordinates xi, yi. This parameter can be compared to the image of the eye taken with a calibrated microscope, which allows to transform the coordinates xi, yi into absolute coordinates.

Alternatively to calculating the Fourier transform of a dataset derived from ri(xi, yi), ray tracing can be used for determining at least one parameter of the eye, such as one or more parameters describing the PSF of the eye.

Such ray tracing can be based e.g. on the following steps:
Measuring, by means of OCT, the geometry of least some of the refractive structures of the eye. Advantageously, this includes measuring the geometry of the anterior and posterior surface of cornea 36, anterior lens surface 40, and posterior lens surface 42.
Using ray tracing, taking into account the geometries measured by the previous step, to calculate the intensity distribution at the location of retina 44 generated by the superposition of a plurality of ideal beams parallel to direction D: In a raytracing simulation, a set of parallel and evenly distributed beams can be assumed, that covers the cornea of the measured eye. The trajectory of each beam is calculated as it passes through the eye until it reaches the retina, by calculating the new beam axis caused refraction at each optical interface (anterior and posterior cornea, anterior and posterior lens) using Snell's law and refractive indices known from literature (e.g. the eye model of Le Grand, values can be found in Atchison D A and Smith G, “Optics of the Human Eye”,). If sufficient beams are used in this simulation, the density distribution of the points where those beams cross the retinal surface provides a good approximation of the PSF of the eye for the axis of incidence of the simulated beams.

For each simulated beam, a transmission value is determined based on one or more of the reflection values ri, assuming that the reflection values ri are e.g. proportional to the transmission at the points xi, yi, and xi, yi are in vicinity of the coordinates of the simulated beam (e.g. having a distance of less than 10 spot sizes). This transmission value ri (or a composite value r′i) can be used as weighting factor for that particular simulated beam. The PSF resulting from such a simulation represents the optical imaging quality of the eye including effects of aberrations and obstructions (scattering and/or absorption).

The simulation can be further improved by taking into account the angle of incidence of each beam with respect to the retina and weighting each beam according to the Stiles-Crawford effect (Stiles and Crawford 1933), i.e. the angle-dependence of the retinal sensitivity.

Techniques for carrying out such ray tracing calculus are e.g. described in:
1) Spencer G, Murty M, “General ay-Tracing Procedure, Journal of the Optical Society of America, Vol. 5, Issue 6, Page 672 (1962), DOI: 10.1364/JOSA.52.000672
2) Einighammer J, “The Individual Virtual Eye”, Dissertation at Univ. Tübingen (2008), Chapter 3.2.3, http://hdl.handle.net/10900/49149, and references therein.
3): Einighammer J et al., “The individual virtual eye: a computer model for advanced intraocular lens calculation”, J Optom 2009; 2:70-82, https://doi.org/10.3921/joptom.2009.70 and references therein.

The eye's PSF can e.g. be directly displayed to the operator using graphs as shown in FIG. 6 or 7. Alternatively or in addition thereto, the mathematical convolution of the PSF with a given image can be calculated and displayed in order to visualize how the eye sees the given image.

Notes

Advantageously, the A-scans used for measuring the parameter includes plurality of A-scans, advantageously at least 10 A-scans, in particular at least 100 A-scans, that have mutual distances, at the cornea, of at least 1 mm, i.e. a macroscopic region of the eye is examined.

In particular, the plurality of A-scans is distributed over the whole pupil of the eye, which allows to measure the transmission over the whole pupil. The distribution can be even or irregular. Advantageously, it has a resolution of at least ten points horizontally (i.e. along x) as well as vertically (along y).

In the embodiments above, the A-scans i all have the same direction of incidence, i.e. they are, before entering the cornea, parallel to direction D, which is advantageously parallel to the optical or visual axis A of the eye.

In another embodiment, probe beams having different directions of incidence can be used.

For example, a first plurality of A-scans for probe beams having mutually parallel directions of incidence, along a first direction (e.g. D), can be recorded. In addition, a second plurality of A-scans for probe beams having mutually parallel directions of incidence along a second direction (e.g. D′ in FIG. 4) can be recorded. These measurements can e.g. be used for at least one of the following purposes:
The PSF of the eye for differing angles of incidence can be measured.
Information on the z-coordinate of the defects can be obtained by measuring how a structure in the values ri(xi, yi) is offset between the two sets. Again, for example, ray tracing can be used to simulate parallax effects between the two sets of measurements.

In yet another embodiment, the focal position of the probe beams can be varied while recording the A-scans. For example, for a given location xi, yi, at least two A-scans with different focal positions may be recorded. Since the spatial resolution for defects 50a-50f is best at the focal plane of the probe beams, this allows to e.g. focus the measurement on a specific region of the eye and/or to gain more information about the z-position of given defects.

The present techniques can be used with any kind of OCT, in particular for time-domain OCT as well as frequency-domain OCT. Frequency-domain OCT, and in particular swept-source OCT, is, however, advantageous for its ability to obtain an A-scan quickly.

While there are shown and described presently preferred embodiments of the invention, it is to be distinctly understood that the invention is not limited thereto but may be otherwise variously embodied and practiced within the scope of the following claims.

1. A method for measuring at least one parameter indicative of an optical transmission quality of an eye, said method comprising:
recording a plurality of optical coherence tomography A-scans for different cornea locations xi, yi of said eye,
for each of said A-scans, identifying a reflection value ri at the retina of the eye,
determining the parameter(s) using said reflection values ri and said locations xi, yi.

2. The method of claim 1, wherein said plurality of A-scans includes a first plurality of A-scans having mutually parallel direction of incidence.

3. Tic method of claim. 2, wherein said parallel direction of incidence is parallel to an eye's visual axis.

4. The method of claim 2, comprising a second plurality of A-scans having mutually parallel direction of incidence, wherein the directions of incidence of the first plurality differs from the direction of incidence of the second plurality.

5. The method of claim 1, wherein said plurality of A-scans includes a plurality of A-scans that do not overlap at a cornea of the eye.

6. The method of claim 1, comprising focusing probe beams for at least part of said A-scans at an anterior part of the eye.

7. The method of claim 1, comprising focusing probe beams for at least part of said A-scans at an a location between a posterior surface of the eye's lens and the eye's retina.

8. The method of claim 1, comprising varying, while recording said plurality of A-scans by probe beams, a focal position of the probe beams, and in particular wherein for a given location xi, yi, at least two A-scans with different focal positions are recorded.

9. The method of claim 1, comprising displaying said reflection values ri as a function of said locations xi, yi.

10. The method of claim 1, comprising:
performing a Fourier transform on a dataset based on said reflection values ri and
deriving said parameter from a result of the Fourier transform.

11. The method of claim 10, wherein said Fourier transform is a two-dimensional Fourier transform.

12. The methods of claim 1, comprising at least one of:
determining an axial length of the eye from said A-scans by optical coherence tomography, and/or
determining a diameter of the pupil from said A-scans by optical coherence tomography.

13. The method of claim 10 comprising at least one of:
determining an axial length of the eye from said scans by means of optical coherence tomography, and
determining a diameter of the pupil from said A-scans by means of optical coherence tomography
and further comprising using at least said axial length and/or said diameter for estimating an absolute size of a point-spread-function of the eye.

14. The method of claim 1, comprising determining, from said A-scans, a topology of at least one structure of the eye, in particular of the cornea, the iris, an anterior surface of the lens, and/or a posterior surface of the lens.

15. The method of claim 14, comprising: determining the at least one parameter using the reflection values ri and the topology of the structure in ray tracing calculus.

16. The method of claim 1, wherein said optical coherence tomography is Frequency-domain OCT, and in particular swept-source OCT.

17. The method of claim 1, comprising determining, using said reflection values ri, a one- or two-dimensional representation of a point-spread-function of the eye.

18. The method of claim 1, comprising determining, using said reflection values ri, at least one of a location and a spatial extent of absorbing and/or scattering structures in the anterior segment of the eye.

19. An ophthalmologic device comprising
an optical coherence tomography interferometer, and
a control unit structured and adapted to carry out the method of any of the claim 1.

20. The method of claim 18, further comprising representing the location or spatial extend, respectively, as an image in xi-yi-space.

21. The method of claim 1, wherein said plurality of A-scans includes a first plurality of A-scans having mutually parallel direction of incidence as they impinge on the cornea.
